SEQUENCE LISTING (11 sequences)

SEQ ID NO: 1 - DNA, 21 bp, Mycobacterium tuberculosis
cgagctgaacgcgcactgacc

SEQ ID NO: 2 - DNA, 21 bp, Mycobacterium tuberculosis
gcgggtccgaaacgcctctac

SEQ ID NO: 3 - DNA, 26 bp, Mycobacterium tuberculosis
cggatggagcgcctggtcgaaacact

SEQ ID NO: 4 - DNA, 22 bp, Mycobacterium bovis
cgtagtcgtgcagaagcgcaac

SEQ ID NO: 5 - DNA, 22 bp, Myobacterium bovis
ggagcaccatcgtccacatcag

SEQ ID NO: 6 - DNA, 26 bp, Myobacterium bovis
cttggagtggcctacaacggcgctct

SEQ ID NO: 7 - DNA, 23 bp, Myobacterium bovis
catatggagatcactgtctccga

SEQ ID NO: 8 - DNA, 25 bp, Myobacterium bovis
ctaaatgcagtgaattgtgaccaag

SEQ ID NO: 9 - DNA, 26 bp, Myobacterium bovis
tggaagacactgctcccgataatgtg

SEQ ID NO: 10 - DNA, 22 bp, Myobacterium bovis
tggtttggtcatgacgccttcc

SEQ ID NO: 11 - DNA, 22 bp, Myobacterium bovis
ggagcaccatcgtccacatcag

(19) United States
(12) Patent Application Publication
(10) Pub. No.: US 2010/0304371 A1
(43) Pub. Date: Dec. 2, 2010

(54) COMPOSITIONS AND METHODS FOR DETECTING MYCOBACTERIA

(76) Inventors: Ana Maria Zarraga, Valdivia (CL); German Rehren Schweppe, Valdivia (CL)

Correspondence Address: FISH & RICHARDSON PC, P.O. BOX 1022, MINNEAPOLIS, MN, 55440-1022, US

(21) Appl. No.: 12/475,453

(22) Filed: May 29, 2009

(51) Int. Cl. C12Q 1/68; C07H 21/00

(52) U.S. Cl. 435/6; 536/23.1; 536/24.33

(57) ABSTRACT

The present invention features compositions and methods that can be used to analyze samples for the presence of mycobacterium from MTC, M. bovis and M. tuberculosis. Moreover, the methods can be used, for example, to differentiate M. bovis from M. tuberculosis and other MTC specimens. The compositions include oligonucleotides that can be used as primers and probes. The primers can be used in multiplex PCR, providing specificity and sensitivity even when used in single run amplifications.

### TECHNICAL FIELD

The present invention relates to compositions, including products, systems, and kits, and methods for identifying, detecting, or differentiating among species of mycobacterium such as Mycobacterium bovis and Mycobacterium tuberculosis. The compositions include nucleic acids, which can be used to detect mycobacteria in biological samples using real-time PCR techniques.

### BACKGROUND

The Mycobacterium genus includes parasites, saprophytes and opportunist pathogens. This is a varying group of gram-positive bacteria, some of which are pathogens that cause serious diseases in human beings and animals.

The group of bacillus that causes tuberculosis in mammals (human and animals) is called Mycobacterium tuberculosis complex (MTC), which comprises highly related mycobacteria exhibiting high homogeneity in nucleotide sequence in spite of their differences in pathogenic strength, geographic distribution, epidemiology, preferred host and certain physiologic characteristics such as colony morphology, resistance patterns, and susceptibility to inhibitors. Some of these mycobacteria are considered etiologic agents of human tuberculosis, such as M. tuberculosis, M. bovis, Mycobacterium africanum (subtypes I and II), bacillus Calmette-Guérin (BCG), and Mycobacterium microti.

The genomic sequence of M. bovis is more than 99.95% identical to that of M. tuberculosis. No genes unique to M. Bovis have been found, implying that differential gene expression is what predisposes the attraction to the human or bovine. Then, even though the species are related, they can be differentiated by their host, their virulence in humans, and physiological characteristics (Heifets and Good, 1994).

The natural habitat of MTC species is infected human and other animal tissues. Mycobacterium bovis causes tuberculosis in cattle, humans and other primates, as well as other animals like dogs, cats, pigs, and parrots. Close to 10% of the cases of human tuberculosis worldwide are due to M. bovis. There is a correlation between eradication of tuberculosis in cattle and the prevalence in humans. In industrialized countries, the control of disease in cattle and pasteurization of milk have drastically reduced the incidence of infection caused by M. bovis. However, zoonotic tuberculosis is still present in developing countries, where control and surveillance on bovine tuberculosis are inappropriate or they are not applied, and domestic, wild animals and cattle share pasture zones (Cristina Prat y colab. SEIMC).

Tuberculosis is primarily a bovine pathogen. Transmission of this pathogen may occur by inhalation, ingestion, or through physical contact. When the transmission route is by ingestion or handling of contaminated material such as non-pasteurized milk, cervical lymphadenopathy (scrophula), intestinal or skin lesions commonly occur.

For detection and differentiation of M. bovis from M. tuberculosis and members of MTC (Haas et al. 1997; and Niemann et al. 2000), classic techniques such as biochemical, phenotypic, and growth tests are used. Unlike M. tuberculosis, niacin production and nitrate reduction tests are negative in M. bovis, however, other biochemical characteristics are indistinguishable (Metchock B. G. et al. Manual of Clinical Microbiology. ASM Press, Washington D.C., 1999: 399-437). The application of molecular biology techniques to identify mycobacteria in liquid or solid medium allows faster and more reliable diagnostics.

Most of the commercialized techniques are based on polymerase chain reaction (PCR) amplification of specific gene sequences of Mycobacterium genus species useful for distinguishing MTC members from other mycobacteria. Molecular techniques are not currently used in many places for diagnosis in humans. This may be due to the demands of a highly sensitive technology and the availability of laboratories with personnel and infrastructure sufficient to guarantee reliable diagnosis. In Chile, conventional PCR for the diagnosis of bovine tuberculosis is being used by the Reference Laboratory of the Agriculture and Livestock Service (Servicio Agricola y Ganadero, SAG). This method, disclosed in Chilean Patent No. 42,351, was developed to provide a quick, sensitive and specific diagnosis for MTC bacteria, specifically M. bovis. This PCR test is different from the methods proposed in the present application.

The molecular techniques commonly used for tuberculosis diagnosis are:

hybridization techniques. The most frequently used sequence is the ribosomal gene sequence which codes for 16S rRNA, which is well conserved but it has variable zones with gender and species-specific nucleotide sequences, which are the ones that are amplified. This sequence is the target for AccuProbe® DNA probes (Gen Probe, bioMerieux, WO 8402721, WO 8803957).

Amplification and hybridization techniques with specific probes. The GenoType system (Hain Lifescience) uses another sequence that also contains conserved and variable regions, codifying for rRNA 23S. The Inno-LiPA® Mycobacteria system (Innogenetics) combines a PCR technique designed to amplify the rRNA 16S-23S spacing region (ITS, internal transcribed spacers) which shows more variability, followed by reversed hybridization onto nitrocellulose stripes using specific probes for various different species.

Amplification and digestion with restriction enzymes. The hsp 65 gene (codifying for HSP protein of 65 kD (HSP heat shock protein)) is the most frequently amplified by the PRA system (PCR restriction enzyme analysis) in which the PCR reaction is followed by digestion with two restriction enzymes and the fragments obtained are detected in agarose gels following ethidium bromide staining The resulting restriction patterns are specific for the different species of mycobacteria.

PCR amplification. The Gen-Probe® kit commercialized as “Amplified® M. tuberculosis direct test” kit or MTD test kit (Gen-Probe Inc. San Diego, Calif., USA), amplifies specific MTC rRNA, followed by amplicon detection according to the Gen Probe HPA method (Hybridization Protection Assay).

Sequencing. This technique is based on the amplification and subsequent sequencing of a fragment of some amplified genes. This technique has allowed the description of new mycobacterium species, particularly from 16s rDNA analysis.

From the phenotype and genotype identification as MTC, sensitivity studies (using BACTEC system, Beckton Dickinson) that show pyrazinamide resistance will only result in a presumptive identification of M. bovis. PCR techniques have also been developed for point mutation detection in pyrazinamidase enzyme gene (pncA), which is absent in M. Bovis, giving rise to pyrazinamide resistance. Nonetheless, M. bovis subtypes sensitive to pyrazinamide have been described.

Diverse molecular methods have been evaluated for the identification of different species that form MTC. PCR based techniques followed by RFLP analyses (restriction fragment length polymorphism) is the referential method for M. tuberculosis individual strain identification; IS6110 insertion sequence, highly conserved in mycobacteria DNA belonging to MTC, shows numerous copies in M. tuberculosis chromosome (10-20), in variable locations, while M. bovis is characterized by containing only between one and six copies of IS6110 fragment. The use of these techniques alone is not efficient for taxonomic classification of MTC members. Strains with little IS6110 number of copies must be typified by spoligotyping technique (spacer oligonucleotide typing), which detects the absence or presence of variable spacing sequences within the DR region (direct repeat locus). The spacers within the DR region are amplified by PCR and detected by hybridization of the biotin labeled PCR product with oligonucleotides derived from the spacing sequences attached to a membrane. Typically, M. bovis does not contain spacers 39 to 43. With this technique it has been possible to detect species sensitive to pyrazinamide, named M. bovis caprae subtype, which is recognized by its absence of spacers 3 to 16 as a spoligotype pattern. A resistant subtype has been named M. bovis bovis subtype.

Discrimination capacity by spoligotyping is still limited. PCR-RFLP of genes such as gyrA, katG, pncA, oxyR, hsp65 y gyrB has been more useful, especially polymorphism analysis of gyrB sequence. Recently, Huard et al. (2003) have developed a PCR typifying panel for MTC that detects “diversity regions” within several chromosomal loci representing genomic suppressions that allow distinction among different sub species. For example, the absence of gene Rv1510 allows differentiation between M. bovis and M. bovis BCG from other subspecies. Using primers for different regions at the same time, it is possible to obtain an amplification pattern for each species. Neither this technique nor the polymorphism analysis of gyrB allows differentiation between M. tuberculosis and M. africanum subtype II.

A previous comparative study of M. bovis and M. bovis BCG using genomic subtractive hybridization showed three regions, named RD1, RD2 and RD3, that were not present in M. bovis BCG compared to M. bovis (Mahairas et al, 1996). Similarly, other studies of genomic differences between M. bovis, M. bovis BCG and M. tuberculosis have shown the existence of many locations of polymorphisms in these strains (Philipp et al., 1996). Additional analyses have revealed that one polymorphism was produced by deletion of 12.7 kb in M. bovis and BCG compared to M. tuberculosis (Brosch et al., 1998). Therefore, apparently there are two types of deletions; those that are absent in BCG but are present in M. bovis and M. tuberculosis, and those that are absent in M. bovis and BCG but present in M. tuberculosis. Complementary to studies carried out by Mahairas et al. (1996) (see U.S. Patent Application Publication No. 2005/0250120) the inventors identified 10 loci that were absent in M. bovis BCG compared to M. tuberculosis. It was found that three of those specific deletions were identical to those previously described RD1, RD2 and RD3. Maintaining the nomenclature, seven other deletions in the genome of M. bovis BCG/M. bovis, were then named RD4, RD5, RD6, RD7, RD8, RD9 and RD10. Similarly, Gordon et al., 1999 and Behr et al., 1999, described 14 regions, named RD1 to RD14, that were absent in M. bovis BCG but present in the genome of M. tuberculosis H37Rv.

In WO 2007/028414 a method and a combination of nucleotidic sequences of RD9 and IS6110 are described in order to distinguish M. tuberculosis and M. canetti from other mycobacteria of MTC.

Taylor et al. (2007) performed a fast detection of M. bovis DNA using PCR. For that purpose, they used the multicopy insertion element IS1081 present in the M. tuberculosis complex, and to confirm M. bovis they used primers flanking the RD4 region. It was determined that IS1081 could work adequately. Nonetheless, methods using single copy markers in the genome, like RD4, do not operate well in these methods and require improvement in order to become a viable method test. In this respect, the present application has developed a different combination of primers and probes appropriate for amplification of the gene target RD4 and IS1081 by real time PCR amplification (TaqMan type). In this document of the previous art (Taylor 2007), preliminary information is provided with regards to real time PCR using SYBRGREEN fluorophore that intercalates the DNA double helix regardless of its sequence and emits fluorescence with some low degree of specificity. Unlike the previous art, the TaqMan real time PCR system of this invention uses different sequence-specific primers, a fluorophore labeled probe that specifically hybridizes with the target sequence to be amplified and provides greater sensitivity and specificity. Taylor describes a sensitivity of 91% for IS1081 and 59% for RD4.

The use of real time PCR (fast cycling PCR, QPCR) which is equipped with a temperate air system and therefore exhibits considerably lower transition times compared to a conventional PCR, produces a substantial decrease in detection time of the mycobacterium, for example, by PCR of isolated genetic material from mycobacterium (Chapin and Lauderdale, J. Clin. Microbiol. (1997) 35: 2157-2159). Moreover, the fluorescent measurements represent a method for quick, sensitive and quantitative detection of genetic sequences by using color labeled hybridization probes, particularly with QPCR. Nevertheless, until now it has not been possible to provide a QPCR test to specifically detect members of the M. tuberculosis complex while simultaneously distinguishing M. tuberculosis from other members of the M. tuberculosis complex.

In U.S. Patent Application Publication No. 2007/0015157, a method for the specific detection of M. tuberculosis in a biological sample is described in which nucleic acids amplification is performed using primers convenient for the amplification of a DNA fragment of SEQ ID NO:1, where the aforementioned sequence comprises a fragment of the narGHJI nitrate reductase operon region, the DNA fragment comprises position -125 in the 5′ to 3′ direction, from the translation GTG of the narGHJI operon of the nitrate reductase.

### SUMMARY OF THE INVENTION

Considering the above mentioned facts, it is necessary to provide a diagnostic tool that may work directly on biological samples of mammals and discriminate among mycobacteria. Such tools are the specific subject of the present invention, which provides methods and the means for a rapid, sensitive and specific detection of M. tuberculosis and M. bovis and their distinct identification, one from the other. More specially, the present invention provides an improved assay with respect to sensitivity, precision and specificity toward M. bovis, which is advantageous when the pathogen is found in only few copies in the analyzed biological samples.

### BRIEF DESCRIPTION OF THE FIGURES

The following figures are related to the present invention, correspond to evidences and illustrate some embodiments of the present invention. These figures are not intended to restrict in any way the scope of the present invention.

FIGS. 1(A)-1(C) are photographs of gels run to examine pairs of primers that can be used in the present methods, according to the size and specificity of the obtained PCR products. The result of the combination of different primers designed for the genetic targets included within the invention are shown. (A) corresponds to the genetic target IS1081, characteristic of mycobacteria from the tuberculosis complex. In each lane the combination of primers used is indicated. (B) corresponds to the genetic target RD4, specific for M. bovis. In each lane, the combination of primers used is indicated. (C) corresponds to different regions of the genome of Bos taurus evaluated as a genetic target for its use as an internal control. TBB: tubuline; PFKM: muscle phosphofructokinase; TFRC: transferrin receptor. In (A) and (B) M. Bovis gDNA template was used. In (C) bovine gDNA was used. Primers were used in a final concentration of 0.3 μM. In every case, a band corresponding to the expected size was detected.

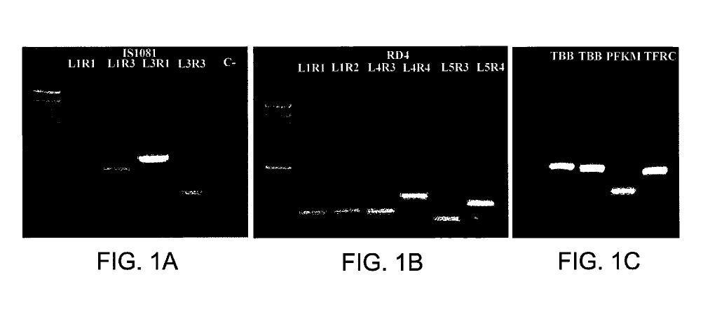

FIGS. 2(A) and 2(B) are photographs of gels showing how the Bos taurus genetic targets previously evaluated as an internal control (FIG. 1(C)) were assayed, with the gDNA indicated on the upper part of each lane as a template (bovine, human, mycobacterial). Non-specific amplification of mycobacterial gDNA was detected for genetic targets TBB-1, TBB-2 and PFKM. As expected, no amplification product was observed for TFRC, which can be used as an internal control. Primers were used in a final concentration of 0.3 μM.

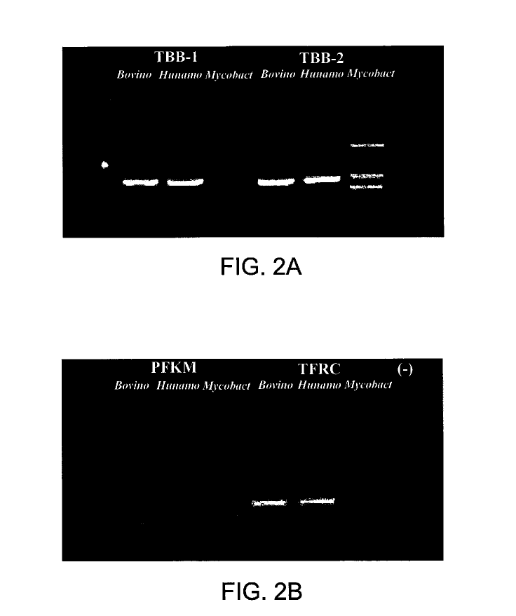

FIGS. 3(A) and 3(B) are photographs of gels. In (A), a new pair of primers was designed within the TFRC genetic target region, Control-L and Control-R, whose 244 by product is observed in the gel and corresponds to the region finally amplified in the present invention. Lanes: 1. human gDNA; 2. bovine gDNA (negative sample in culture); 3. bovine gDNA (positive sample in culture); 4. negative control. Primers were used at a final concentration of 0.3 μM. (B) Using the product sequence previously amplified with primers Control-L and R, the TMC probe was designed. Its use as primer (without fluorescent label) together with Control-R, originates a specific product of 175 by (indicated with an arrow). Lanes 1 to 3: primers Control-L and R; lanes 4 to 6: primers Control-R and TMC. Lanes 1 and 4: human gDNA; lanes 2 and 5: bovine gDNA; lanes 3 and 6: negative control without DNA. Primers and probes designed for the TFRC target proved to be functional for both human gDNA and bovine gDNA.

FIGS. 4(A) and 4(B) are amplification curves and FIG. 4(C) is a photograph of a gel. The experiment examined the differential detection of M. bovis and M. tuberculosis. In (C), lane 1 carries M. tuberculosis assayed with primers and probes for M. Tuberculosis complex; lane 2 carries M. bovis assayed with primers and probes for M. Tuberculosis complex; lane 3 carries M. tuberculosis assayed with primers and probes for M bovis; and lane 4 carries M. bovis assayed with primers and probes for M. bovis. In (A) and (B), 530 nm fluorescence is shown (probe for tuberculosis complex) and 640 nm fluorescence (specific probe for M. bovis), respectively.

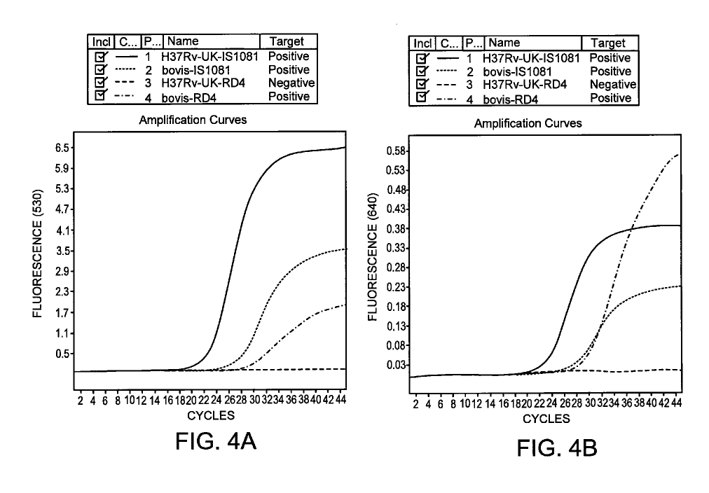

FIG. 5 is a photograph of a gel showing the results of a multiplex type PCR using pairs of primers in the present invention. In lanes 1, 2 and 3, genetic targets are amplified, producing three products of different sizes (marked and indicated with arrows). Lanes 4, 5 and 6 show three genetic targets. The amplified products of two are equal size and the third one is of a different size (indicated with arrows). Lanes 1 and 5: bovine gDNA mixed with M. bovis gDNA; lanes 2 and 4: bovine gDNA; lanes 3 and 6: negative control without DNA. Primers were used at a 0.3 μM final concentration. A 171 by fragment can be amplified using primers L1 and R1, corresponding to target IS1081 (see FIG. 1(A)); amplification of a 174 bp fragment, using L5 and R4 primers, corresponding to RD4 target (see FIG. 1(B)); and the amplification of a 244 bp fragment, using Control-L and Control-R primers, corresponds to TFRC target (see FIG. 3A). The design of the TMT1 probe was based on the sequence of the fragment amplified by primers L1 and R1 (IS1081). The design of TMT2 probe was based on the sequence of the fragment amplified by primers L5 and R4 (RD4).

FIG. 6 is a photograph of a gel run to help determine the assay conditions necessary for an optimal annealing temperature. Multiplex PCR with temperature gradient was used.

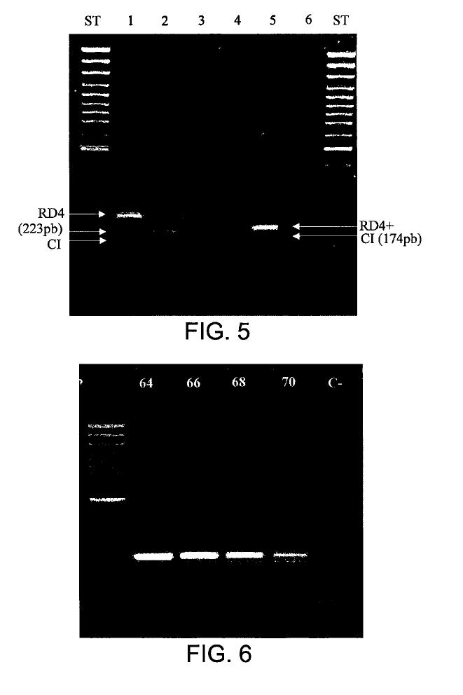

FIG. 7(A) is an amplification curve and FIG. 7(B) is a standard curve from experiments to determine sensitivity for genetic target detection, and in particular the limit of detection of the genetic target IS 1081. In (A), samples numbered 1 to 6 correspond to serial dilutions from 10⁵ to 1 copy from the M. Bovis genome. Samples numbered from 7 to 12 correspond to the same dilutions, with bovine gDNA added to a constant final concentration of approximately 100 ng. Samples 13 and 14 correspond to positive (10⁶ copies of M. Bovis gDNA) and negative controls, respectively. The assay was performed following the established protocol according to the present invention named BoviMan Kit. In (B), the standard curve was obtained from the Cp value of each dilution. It indicates the linear detection range and allows the quantification of the bacterial genomic copies present in the sample. These results demonstrate that the present methods allow the detection of up to 1 equivalent of the genome using pure M. bovis gDNA.

FIG. 8(A) is an amplification curve and FIG. 8(B) is a standard curve from experiments to determine sensitivity for the detection of genetic targets, and in particular the limit of detection of the RD4 genetic target. In (A), samples numbered from 1 to 5 correspond to serial dilutions of M. bovis genome from 10⁵ until 10 copies, with bovine gDNA added in a constant final concentration of approximately 100 ng. Samples numbered from 7 to 11 correspond to the same serial dilutions of M. bovis genome, without bovine gDNA. Samples 6 and 14 correspond to a negative control with bovine gDNA and to a negative control without template, respectively. The assay was performed following the established protocol according to the present invention named BoviMan kit. In (B), a standard curve is obtained from each dilution Cp. It indicates the detection linear range and allows the quantification of the number of copies of bacterial genome present in the sample. The assay allows the detection of 10 equivalents of the genome, using pure M. bovis gDNA.

FIG. 9 is a photograph of a gel showing the presence of M. bovis genome in tissue samples. The assay corresponds to a multiplex PCR (in duplex format) where primers and a probe for the IS1081 genetic target were used, and primers and a probe for an internal control were used. Lanes: 1. M. bovis gDNA; 2. sample of positive tissue; 3. sample of negative tissue; 4. negative control without DNA. The band for IS1081 is observed in lanes 1 and 2, and the band of the internal control in lanes 2 and 3.

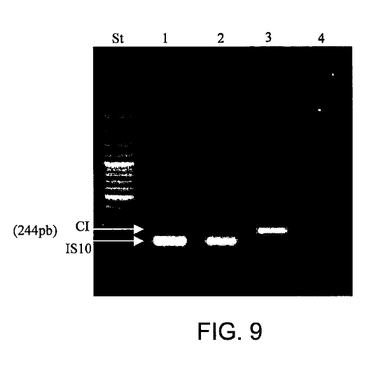

### DETAILED DESCRIPTION

The present invention relates to compositions and methods that can be used for the identification of mycobacteria and the differentiation of M. bovis, which produces bovine tuberculosis, from other mycobacteria of the MTC, such as Mycobacterium tuberculosis, which produces human tuberculosis. The methods can be carried out by real time PCR, allowing fast and direct evaluation of bovine and human tuberculosis pathogens in any materials, including biological samples such as fresh tissue. The nucleic acids targeted by the present methods include those known as RD4 and IS 1081.

In one embodiment, the invention features isolated oligonucleotides that consist of or comprise a designated sequence represented by one of SEQ ID NOs. 1-11. The oligonucleotides are “isolated” in the sense that they are separated from the nucleic acid sequences with which they are normally contiguous in a naturally occurring cell. The oligonucleotides can comprise the sequence of any of SEQ ID NOs. 1-11 and can include (e.g., at the 5′ terminal, the 3′ terminal, or both) additional sequence, which may hybridize with the target sequence or may be heterologous. For example, the oligonucleotides can include 1-10 (e.g., 1, 2, 3, 5, or 8) additional nucleotides at the 5′ terminal, the 3′ terminal, or both.

The oligonucleotides can be used as conventional primers or probes, and the methods described herein can include one or more steps directed toward identifying a patient or material in need of testing; providing a sample (e.g., a sample of biological tissue); processing the sample (e.g., extracting genetic material from the sample); amplifying a target sequence within the sample/material; detecting an amplification product; and conveying the results of the analysis to the patient or some other person (e.g., a health care provider or public health service). The present methods are particularly advantageous given how difficult it is to cultivate these pathogens and that the pathogen or gene target may be present in limited amounts in a given sample.

The present methods can be carried out using a conventional PCR assay, and the product(s) can be assessed by visual analysis of the length of the generated fragment following electrophoresis and classical staining procedures. Unlike the present methods, some PCR techniques previously described allow the identification of bacteria from M. tuberculosis complex, but they cannot distinguish between the human and animal pathogen, particularly at low copy number, with high sensitivity.

As noted, compositions useful in the methods described herein are also within the scope of the present invention. These compositions include nucleotide sequences that can be used as primers and/or probes to identify the evaluated species. Preferably, the invention includes amplification primers or hybridization probes for identification of MTC organisms, particularly M. tuberculosis, and M. bovis, wherein those primers have as their targets IS 1081 and RD4 sequences, respectively, and wherein those primers do not cross react with any other nucleic acid from mycobacterium. Moreover, the invention comprises primers and/or probes that can be used as internal controls. These sequences can be oligonucleotides derived from a nucleotide sequence or gene belonging to the eukaryotic host. The primers can be used in pairs with a corresponding probe applied to the amplified sequence. Preferably, the primers and probes that generate the internal control will not interfere with the correct performance of the test primers and probes. The oligonucleotides that target a mycobacterial-specific sequence and the oligonucleotides that are useful as internal probes can be formulated in the same manner (e.g., provided in a lyophilized form or in solution) and packaged together in kits. In a preferred embodiment, the internal control can be the transferrin receptor, TFRC.

In one embodiment, the invention comprises a set of primers and probes selected from at least three sequences of SEQ ID No 1 to SEQ ID No 11, preferably a set of primers and its corresponding probe, more preferably two sets of primers and their respective probes, and still more preferably three sets of primers and their respective probes. For example, the present compositions can include three pairs of primers and the three hydrolysis probes corresponding to each set of primers. One probe can be specific for RD4, another probe can be specific for IS1081, and a third probe can be specific for the internal control sequence. Optionally, the compositions can include two pairs of primers and their corresponding hydrolysis probes, one of the probes being specific for RD4 or for IS1081 and the other probe being specific for an internal control sequence. The primers and probe represented by SEQ ID NOs 1-3 are designed to amplify and detect a 171 by region, corresponding to an IS1081 insertion sequence present in all species of the M. tuberculosis complex. The primers and probe represented by SEQ ID NOs 4-6 or 10-11 are designed to amplify and detect a region of 174 by or 223 bp, respectively, corresponding to a region flanking an RD4 deletion. This deletion is specific for M bovis and the PCR product is obtained only in the presence of this deletion. Probe 2 hybridizes exactly on the borders (or edges) of the deletion. The third pair of primers and probe, which are represented by SEQ ID Nos. 7-9, amplify a region of approximately 200 by corresponding to the TFRC eukaryotic gene (transferrin receptor), both in human and bovine DNA. The function of this pair of primers and probe is to provide an internal control of the amplification (controls the quality of DNA extraction, the accomplishment of the detection method and the presence of reaction inhibitors). The aforementioned sequences may be used together (as multiplex, duplex, triplex or more) or in separate or individual forms. Consequently, the assay is capable of differentiating with high specificity, sensitivity and efficiency, in only one step, M. bovis from M. tuberculosis and from the other members of the M. tuberculosis complex.

Primer and probe sequences according to the invention correspond to the following:

SEQ ID NO: 1:
5′ CGAGCTGAACGCGCACTGACC 3′ (T1L1)
SEQ ID NO: 2:
5′ GCGGGTCCGAAACGCCTCTAC 3′ (T1R1)
SEQ ID NO: 3:
5′ CGGATGGAGCGCCTGGTCGAAACACT 3′ (TMT1)
SEQ ID NO: 4:
5′ CGTAGTCGTGCAGAAGCGCAAC 3′ (T2L5)
SEQ ID NO: 5:
5′ GGAGCACCATCGTCCACATCAG 3′ (T2R4)
SEQ ID NO: 6:
5′ CTTGGAGTGGCCTACAACGGCGCTCT 3′ (TMT2)
SEQ ID NO: 7:
5′ CATATGGAGATCACTGTCTCCGA 3′ (C-L)
SEQ ID NO: 8:
5′ CTAAATGCAGTGAATTGTGACCAAG 3′ (C-R)
SEQ ID NO: 9:
5′ TGGAAGACACTGCTCCCGATAATGTG 3′ (TMC)
SEQ ID NO: 10:
5′ TGGTTTGGTCATGACGCCTTCC 3′ (RD4L4)
SEQ ID NO: 11:
5′ GGAGCACCATCGTCCACATCAG 3′ (RD4R4)

The present invention further comprises a PCR amplification method in biological samples, preferably in real-time PCR where it is specially adapted to perform a multiplex-type PCR, either in one single tube for both targets or in two or more tubes for each specific target, and may be duplex PCR with two targets or one target and one internal control, or triplex PCR with two targets and one internal control, providing the results, for example, in a single run amplification. Such method is used for the detection and differentiation of M. bovis from M. tuberculosis or, mycobacteria from MTC in biological samples.

Notwithstanding the above, primers and probes according to the present invention, together or individually, may be applied and be useful in other PCR assays and procedures, hybridization-based techniques, and quantitative and qualitative recognition systems known in the art. The samples may be minimally processed or processed more extensively by, for example, previous conditioning procedures, purification or extraction. The sample can be obtained from cultures of cells, tissue or extracts thereof, as well as from animals, parts thereof, or samples taken from their tissues and/or fluids. The human or other animal from which a sample is taken may be sick or apparently healthy (e.g., one may only suspect infection and/or contagion).

Preferably, the invention uses sets of oligonucleotides comprising at least three of the sequences described above. However, additional oligonucleotides (e.g., six or nine in all) can be used to assess a given sample. As noted, the combinations of oligonucleotides may include hydrolysis probes. The oligonucleotides can be mixed, and the probe sequence can be included in the same proportion as the respective pairs of primers. The sequences of the invention are selected from the sequences SEQ NO 1 to SEQ NO 11 described above.

The specific sequences of the invention enable the detection of mycobacterium from MTC, differentiation of M. bovis from M. tuberculosis and other MTC specimens, as well as primer designed to be used in multiplex PCR, providing specificity and sensitivity even when they are applied in single run amplifications. The following table provides a summary of how various results would be interpreted when applied in accordance with the multiplex form.

TABLE 1

| PCR Result | Meaning of the assay |
|---|---|
| RD4+; IS1081+ | MTC Mycobacterium with presence of M. bovis. |
| RD4−; IS1081− | No MTC mycobacteria or M. bovis. |
| RD4−; IS1081+ | MTC Mycobacterium. |
| RD4+; IS1081− | Unlikely option because both targets are in M. bovis; |
| | it is a false positive for M. bovis. However, this |
| | outcome may indicate procedural errors, problems |
| | with reagents, or operation failures. |
| TFRC | Internal control is a quality indicator of extraction and |
| | detection procedures. |

In another embodiment, the present invention provides compositions comprising such sequences as a group or individually. Preferably, such compositions comprise at least one pair of primers, more preferably, at least two pairs of such primers, more preferably, comprise three pairs of primers, wherein such composition preferably may comprise respective hydrolysis probes, in the same proportion as the referred pairs of primers, however, probes may, either as a group or individually, take part in a different composition. It will be understood that from a composition comprising the SEQ ID NO:1 individually to those comprising 11 sequences altogether fall within scope of the invention, taking into consideration possible and functional combinations.

The probe sequences of the invention may be adapted to different formats known by those skilled in the art, including Taqman™, Scorpion™ probes or those called beacon probes. Preferably, Taqman™ probes are used in the invention.

According to the invention, the probes can comprise a detectable label such as a fluorophore and/or a quencher, and both of these types of elements are known in the art. Examples of known fluorophores, which can be used here alone or in combination, include FAM, DYXL, Hex, Tet, Joe, Rox, Tamra, Yac, Max, Edans, Cy5, LC670, Fluorescein, Coumarin, Eosin, rhodamine, Bodipy, Alexa, Cascade blue, Yakima yellow, Lucifer yellow, and Texas red. The probes can include fluoropheres emitting different wavelengths. Preferable fluoropheres include FAM and DYXL. The quencher can be any of those known in the art that are compatible with the fluorophore. BBQ is a useful quencher.

In a related embodiment, the invention comprises PCR combinations comprising at least a pair of primers and/or probes, or the aforementioned compositions according to the invention. Such PCR combinations, preferably comprise the means of the invention such as primers, probes, dilution solutions and the like; as a whole or separately; enabling the amplification and detection of RD4 and/or IS1081 jointly, successively or separately. Such PCR combinations may be dry, in a solution, lyophilized, or such common forms known in the art. It will be understood that from PCR combinations comprising the SEQ No 1 individually, to those comprising 11 sequences as a whole, fall within the scope of the invention, taking into consideration possible and functional combinations.

At the same time, the invention discloses a kit for specific and highly sensitive detection of M. bovis and/or M. tuberculosis and/or MTC. This kit or parts kit comprises a device and/or container with at least one pair of primers and a probe of the invention, and/or a composition of the invention containing thereof, and/or a PCR combination comprising one of the above. Optionally, according to the target to be detected, the kit comprises means to prepare the reaction, and instructions for the use thereof.

In one multiplex embodiment, the kit comprises the PCR primers, probes, and/or their compositions and/or combinations, for detecting RD4 e IS1081, wherein such products may be individually or as a whole.

A person skilled in the art will know how to identify the necessary components as well as the necessary reagents to perform detection according to the invention.

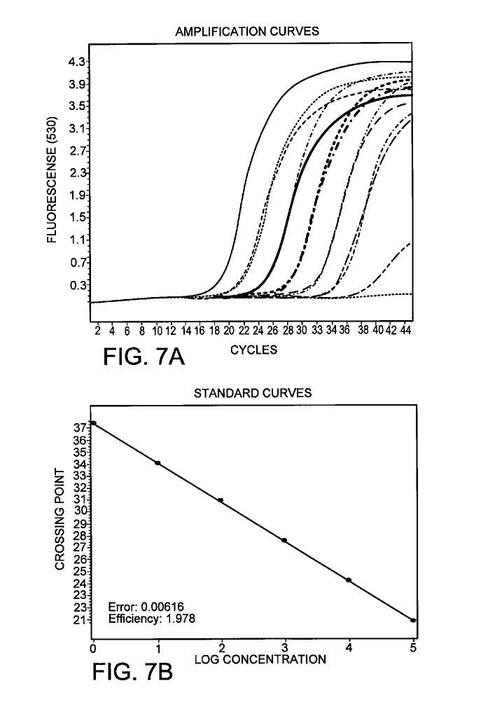

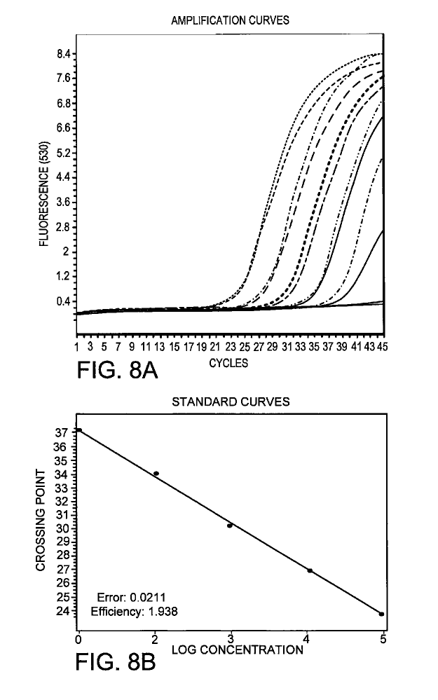

One significant part of the invention consists of providing a procedure for processing samples and extracting genetic material. This is of great value since the microorganism to be detected is generally found in very low count samples. FIGS. 7 and 8 show the method of detection and identification described herein that, according to the invention, enables to identify up to one desired microorganism in the sample for analysis. This is due to the joint qualities of the sequences used for the specificity of the case, as well as to the extraction procedure of genetic material.

The procedure for obtaining genetic material comprises the steps of obtaining the sample, preferably, directly from the animal and more preferably, a nodule, extracting a fraction of tissue between 20-200 mg, homogenizing on ice, preferably between 0-4° C., centrifugation and extracting the supernatant. The above steps may be performed in controlled conditions such as a biosafety room or enclosure. The process is followed by DNA precipitation, centrifugation, maintaining and drying the precipitate, and finally resuspending the precipitate in up to 500 μl of buffer solution. Such solution may be stored at −20° C. until ready for use.

The method of extraction of genetic material preferably comprises transferring nodules to a container and examining the nodule for typical lesions (granulomas). If the center of the lesion is hard (calcification), cut small pieces of tissue that surrounds the hard center, and material from the lesion. Take approximately 50 mg of tissue and cut it in small pieces to facilitate homogenization. Then, the tissue is transferred in a microtube containing beads and about 1 ml of DNAzol is added, stirred into a BeadBeater at high speed, not exceeding 3 minutes, preferably, about 1 min., twice, keeping on ice for at least 1 min. between each stirring. Later, it can be kept at room temperature for approximately 1-3 min. It was centrifuged at about 10,000 rpm for approximately 10 minutes. Then, the supernatant is transferred to a 1.5 or 2.0 ml microtube, taking care not to remove the precipitate or disrupt the beads. If necessary, it can be configured again in conditions equivalent to the above and transferred to another container. Then 0.5 ml of absolute ethanol is added to the volume rescued and mixed by stirring or inversion. This enables to precipitate the DNA, during approximately 10 min., and may be performed at room temperature. Later, it is centrifuged at 4,000 rpm for at least 2 minutes, optionally at about 13,000 rpm for 10 min. at about 4° C. If necessary, the supernatant is removed and the precipitate is washed with about 1 ml of ethanol 75% at least twice and centrifuged each time at 13,000 rpm for 3 min. at about 4° C. Finally, the precipitate is dried at room temperature for at least 10 minutes, taking care not to over dry, and the precipitate is solubilized between 200-400 μl in a TE buffer 0.1× (TE buffer is Tris-HCl 10 mM, pH 8, EDTA 1 mM). If the solution remains cloudy, it may be extracted with a volume of chloroform/isoamilic alcohol (24:1), then centrifuged at about 13,000 rpm for 3 min. at 4° C. and the upper aqueous phase is recovered in a new container.

In a further embodiment, the present invention comprises a method for performing real-time PCR with the samples obtained according to the process of the invention and the aforementioned sequences. This PCR analysis is performed with at least one pair of primers and one probe of the aforementioned sequences, preferably, with two pairs of primers and the corresponding probes, and most preferably at least three pairs of primers and their corresponding probes of the aforementioned sequences are used jointly.

Consequently, a preferred method according to the invention is multiplex PCR.

Such multiplex PCR method may be duplex-, triplex-type or larger. At the same time, the identification of the genic targets IS1081 y RD4, according to the present invention, may be performed in a single reaction or in separate reactions for the genic targets, in the same tube at the same time or successively, or in different tubes, according to the preferred embodiment of the method.

The invention provides a kit for extraction and/or purification of the nucleic acids from the biologic samples to be evaluated. This kit comprises means and solutions used in the aforementioned method for extracting and/or purifying genomic DNA and the instructions for use thereof.

In a further embodiment, the invention provides an integral kit that conjugates means of the kit for M. bovis and/or M. tuberculosis and/or MTC specific and highly sensitive detection, and the kit for extracting and/or purifying nucleic acids from biological samples, both previously described.

In one embodiment, the PCR method of the invention comprises performing real time PCR using the means of the present invention. Preferred PCR method uses necessary equipment and means to carry out this type of assays, preferably, TaqMan system TaqMan (TaqMan LightCycler Kit, available in Roche Laboratories).

The method reaction conditions of the invention are as follows:

TABLE 2

| Phase | Temperature (° C.) | Time (min:sec) | Cycles |
|---|---|---|---|
| Initial incubation | 95 | 10:00 | |
| Denaturation | 95 | 0:10 | 40 |
| Annealing and extension | 68 | 0:12 | |
| Cooling | 40 | 0:30 | |

The amplification and detection method of the invention increases the genic target amplification specificity. According to a preferred embodiment of the invention, sequences and conditions together enable amplification to start, polimerase DNA to hydrolyze from 5′ end releasing reporter fluorophore whose fluorescence is quenched by the fluorescence of the fluorophore at 3′ end (Quencher). After degradation, the fluorophore is separated from the quencher, and fluorescence emission is not absorbed by the quencher, resulting in an increase of fluorescence emission. This process occurs in each PCR cycle, with no interferences in the exponential generation of the amplification product.

Therefore, the present invention incorporates a first level of specificity as for the developed primers flanking the genetic targets disclosed herein, and an additional level of specificity provided by the developed probe which hybridized by specific mating with the center of the genic target. Previous art methods such as SYBRGREEN, do not incorporate such advances because it uses a fluorophore which intercalates between DNA strands unspecifically to double strand sequence.

The specificity of the invention is the result of its primer and probe qualities, as well as the way fluorescent probe is coupled to the aforementioned target amplification process, and to the extraction process of the invention. It should be pointed out that the present invention provides sensitivity of at least 86% both for IS 1081 and for RD4, such samples corresponding to M bovis positive cases, using traditional culture techniques known in the art, thereby confirming the closeness with the currently standard and recognized method. Upon performing such analyses, a lymphatic nodule was used which was divided into two, analyzing one portion through culture, and the other portion through PCR, privileging the culture sample over PCR. This indicates that the test sensitivity of this application could be even greater. The specificity of gPCR of the invention for target IS1081, is 92% in 904 lymphatic nodules from animals not reacting to tuberculin for at least three to five years. This provides evidence of the advantages of the present invention compared to the previous art, particularly, where this parameter was not estimated as in the case of Taylor et al. (2007).

Finally, the preset invention describes the combination of primers, probes, composition, kit, and methods of analysis and extraction for PCR, suitable for amplification and detection of IS1081 and RD4, allowing selectively differentiating MTC species.

### Examples

### Example 1

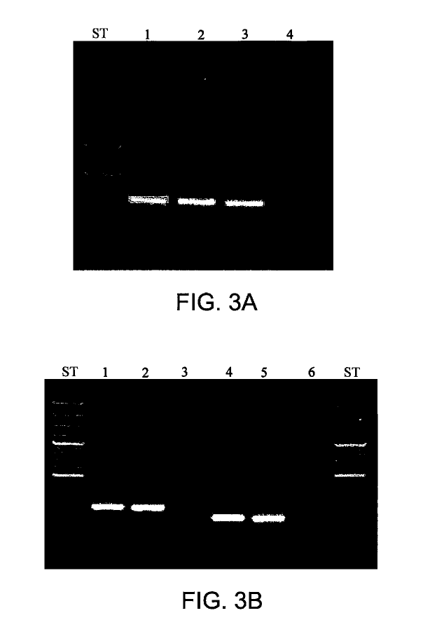

Experiments were carried out to assess the specificity and sensitivity of the methods and sequences described above. To achieve this, the sequences were used in real time PCR reactions to detect M. bovis and M. tuberculosis. As shown in FIG. 3, the sequences used are highly selective in identifying M. bovis.

At the same time, the sensitivity of the method was assessed by determining the minimum quantity of pathogen that can be detected. We assessed a detection limit (i.e., how few copies of the genetic material could be detected). The assay was performed in the presence of an excess (approx. 300 ng) and in the absence of bovine genomic DNA, in order to control the interference in the assay due to the presence of bovine gDNA. As shown in FIGS. 7 and 8, the assay was able to detect, at least, between 1 and 10 copies of the genomic sequence. This range is given by the interference the bovine DNA may present, which is found in variable quantities according to the sample. A range from 10⁵ to 1 bacterial genome was tested, detecting the whole range of quantities.

### Example 2

Genomic DNA was extracted from samples of lymphatic ganglion tissue containing granulomatous lesions (a biosafety cabinet was used). Fifty mg of tissue was cut into small pieces that were transferred to a microtube containing beads, and 1 ml of DNAzol was added. The mixture was stirred in a BeadBeater at high speed for 1 minute, twice, and was maintained on ice at least for 1 min. between each stirring. It was then centrifuged at 10,000 rpm for 10 minutes, and the supernantant was transferred to a microtube of 1.5 or 2.0 ml. Subsequently, the centrifugation was performed outside of the biosafety cabinet. To the recovered volume, 0.5 of absolute ethanol was added, stirred, and the DNA was allowed to precipitate for 10 min. at room temperature. The mixture was centrifuged at 4,000 rpm for at least 2 minutes at 4° C. The supernatant was removed and the precipitate washed with 1 ml of ethanol (75%), twice, washing each time at 13,000 rpm for 3 min. at 4° C. The precipitate was dried at room temperature for 10 minutes and then solubilized in 200-400 ul of a TE buffer 0.1×, centrifuged at 13,000 rpm for 3 min. at 4° C., and the upper aqueous phase was recovered and stored at −20° C. for further use.

The assays were performed following the sample preparation described above and are shown in the appended figures as a reference and as a part of the examples.

### Example 3

According to one embodiment of the invention, a qPCR protocol was performed in accordance with the following reaction mixture:

TABLE 3

| Component | Volume [μl (1X)] | Final concentration | |
|---|---|---|---|
| PCR grade water | 9.6 | | |
| T2 Primer mixture (10 μM) | 1.2 | 0.3 | μM c/u |
| C1 Primer mixture (10 μM) | 1.2 | 0.3 | μM c/u |
| TMT Probe (3 μM) | 1 | 0.15 | μM |
| TMC Probe (3 μM) | 1 | 0.15 | μM |

| | | |
|---|---|---|
| Taqman Master Mix (1 Tube) | 4 | 1X |
| Total | 18 | |

A Kit LightCycler TaqMan Master, a LightCycler Capillaries, a LightCycle cooling block at 4° C. as well as other necessary equipment and materials were used.

The run program consisted of initial incubation at 95° C. for 10 min., 40 cycles of denaturation at 95° C. for 10 seconds and mating-extension at 68° C. for 12 seconds, and a cooling cycle at 40° C. for 0.5 minute.

### Example 4

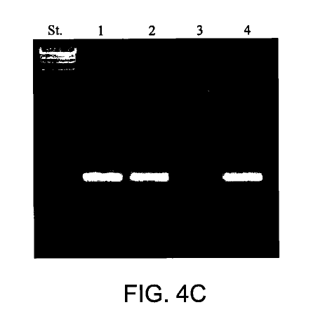

Genomic DNA was obtained from a culture of mycobacteria, M. bovis and M. tuberculosis, and bovine genomic DNA according to Example 2. Subsequently, determination of the conditions described in Table 2 for a multiplex PCR was performed. FIG. 5 shows the results obtained when multiplex assays in a separate-tube duplex format were performed. At the same time, equivalent results (not shown herein) were obtained when multiplex assays in a single-tube triplex format were performed.

What is claimed:

1. An isolated oligonucleotide comprising a sequence represented by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

2. A composition comprising the isolated oligonucleotide of claim 1.

3. The composition of claim 2, wherein the composition is (a) formulated for use as, or for addition to, a polymerase chain reaction mixture or (b) formulated for application to a material comprising amplified nucleic acids.

4. The isolated oligonucleotide of claim 1 further comprising a detectable label.

5. A set of oligonucleotides comprising first and second oligonucleotides that function as primers and a third oligonucleotide that functions as a probe, wherein the first, second and third oligonucleotides each have a sequence represented by one of SEQ ID NOs.1-11.

6. The set of oligonucleotides of claim 5, wherein the first, second, and third oligonucleotides are represented by SEQ ID NOs. 1-3, respectively.

7. The set of oligonucleotides of claim 5, further comprising fourth and fifth oligonucleotides that function as primers and a sixth oligonucleotide that functions as a probe, wherein the fourth, fifth and sixth oligonucleotides each have a sequence represented by one of SEQ ID NOs. 1-11.

8. The set of oligonucleotides of claim 7, wherein the fourth, fifth, and sixth oligonucleotides are represented by SEQ ID NOs. 4-6, respectively.

9. The set of oligonucleotides of claim 7, further comprising seventh and eighth oligonucleotides that function as primers and a ninth oligonucleotide that functions as a probe, wherein the seventh, eighth, and ninth oligonucleotides each have a sequence represented by one of SEQ ID NOs. 1-11.

10. The set of oligonucleotides of claim 9, wherein the seventh, eighth, and ninth oligonucleotides are represented by SEQ ID NOs. 7-9, respectively.

11. A method of detecting mycobacteria in a sample, the method comprising providing the sample and exposing nucleic acids within the sample to a pair of oligonucleotides that specifically target and facilitate amplification of an IS1081 sequence or an RD4 sequence.

12. The method of claim 11, further comprising exposing the nucleic acids to a pair of oligonucleotides that specifically target and facilitate amplification of an internal control sequence.

13. The method of claim 11, wherein the sample is suspected of containing Mycobacterium tuberculosis complex (MTC) mycobacteria, M. bovis or M. tuberculosis.

14. The method of claim 11, wherein the amplification is carried out in vitro.

15. The method of claim 11, wherein the amplification is carried out by the polymerase chain reaction (PCR).

16. The method of claim 15, wherein the PCR is real time PCR.

17. A kit comprising the isolated oligonucleotide of claim 1 and instructions for use.

18. A method comprising
providing a sample of tissue comprising nodules;
examining the nodules for granuloma and/or calcification;
excising small pieces of the tissue surrounding the granuloma or calcification;
homogenizing the pieces of tissue;
transferring the homogenized tissue to a microtube containing beads and about 1 ml of DNAzol;
precipitating the DNA within the tissue;
drying the precipitate;
resuspending the precipitate; and
analyzing the precipitated for the presence of RD4 and/or IS1081 target sequences.
